(19) United States
(12) Patent Application Publication
(10) Pub. No.: US 2010/0023065 A1
(43) Pub. Date: Jan. 28, 2010

(54) TISSUE ACCESS DEVICE WITH ALIGNMENT GUIDE AND METHODS OF USE

(76) Inventors: ANDREA M. WELCH, Plainfield, IL (US); GEORGE T. DELLI-SANTI, Scotts Valley, CA (US); MICHAEL D. FOSTER, Palo Alto, CA (US)

Correspondence Address: ARTHROCARE CORPORATION;ATTN: Matthew Scheele, 7500 Rialto Boulevard, Building Two, Suite 100, Austin, TX, 78735-8532, US

(21) Appl. No.: 12/180,277

(22) Filed: Jul. 25, 2008

(51) Int. Cl. A61B 17/56; A61B 17/28

(52) U.S. Cl. 606/86 R; 606/205

(57) ABSTRACT

An access assembly for use during a medical procedure including accessing a selected bone structure or target tissue, includes a cannula and stylet with an alignment guide removably attached to the cannula. The alignment guide shows the relative alignment between the access assembly and the viewing axis of an imaging system, thereby making placement of the access assembly more accurate. Once a desired alignment is achieved, the access assembly may then be advanced further towards the target tissue until a desired placement is achieved.

### FIELD OF THE INVENTION

The present invention relates generally to an apparatus for accessing bone or tissue structures and, more particularly, to an access assembly that incorporates a radiopaque alignment element adapted to aid in the placement of a cannula within a selected body structure.

### BACKGROUND OF THE INVENTION

A number of apparatus have been developed for accessing target areas of bone or tissue within a patient. Procedures such as vertebroplasty and kyphoplasty require the precise insertion and placement of an access device such as a cannula into a target area of bone to achieve access to an implantation site. In vertebroplasty, cancellous bone of an injured vertebra may then be supplemented with “bone cement,” e.g., polymethylmethacrylate (PMMA) or another material, in order to provide for stabilization of the vertebral body. In kyphoplastly, an expandable device such as a balloon is inserted into the interior of the vertebra and expanded. Following removal of the expandable device, the resulting void is typically filled with bone cement to promote stabilization of the vertebral body. Vertebroplasty and kyphoplasty are desirable from the standpoint that it is minimally invasive as compared to a conventional procedures requiring surgically exposing a tissue site that is to be supplemented with bone cement.

Several procedures are known for accessing a desired site in the cancellous bone of a vertebral body, or substantially any other cancellous bone, to deliver an expandable device and/or bone cement or another suitable hard tissue implant material to stabilize, or build up, a target site as taught by U.S. Pat. No. 6,280,456, U.S. Pat. No. 6,248,110, U.S. Pat. No. 5,108,404, and U.S. Pat. No. 4,969,888, which are each incorporated herein by reference.

To gain access to a hard tissue implantation site, as described in U.S. Pat. Nos. 6,019,776 and 6,933,411, which are each incorporated herein by reference, a straight needle or cannula in combination with a stylet may be employed. As discussed therein, a stylet incorporating self-tapping threads may be utilized to penetrate the cortical bone of the vertebra. Once access is achieved and the stylet is removed from the cannula, bone cement may be delivered through the cannula for the purpose of stabilizing the hard tissue implantation site.

Many access cannula devices incorporate a T-grip handle; however, alternate handle types are also available. For example, Clear-View® needles, manufactured and sold by the assignee, incorporate a barrel-shaped or cylindrical handle instead of a traditional T-grip type handle. The barrel handle of the Clear-View needle provides various advantages including presenting a so-called “bull's eye” image when viewed fluoroscopically. When viewed along the longitudinal axis of the cannula assembly, the outer wall and the interior interlocking assembly of the barrel-shaped handle have sufficient material thickness and radiopaque properties to appear as concentric circles when viewed under a fluoroscope and still allow visualization of the cannula and the target body structure. The concentric circles or “bull's eye” may then be used to gauge the alignment of the cannula with a target body structure. However, the size of barrel shaped handles may present problems in procedures that require the placement of multiple cannulas in a small area or closely juxtaposed, such as a vertebroplasty procedure requiring the bipedicular access of a vertebral body.

### SUMMARY OF THE INVENTION

Therefore a need has arisen for an improved system and method for accessing a bone structure.

A further need has arisen for an improved system for showing the relative alignment of an access device and a radiographic viewing device.

The present disclosure presents an improved access assembly for use during a medical procedure including accessing a selected bone structure or other target tissue, and includes a cannula and stylet with an alignment guide removably attached to the cannula. The alignment guide preferably shows the relative alignment between the access assembly and the viewing axis of an imaging system when viewed fluoroscopically and/or radiographically, thereby making placement of the access assembly more accurate. Once a desired alignment of the access assembly is achieved, the access assembly may then be advanced further towards the target tissue until a desired placement is achieved.

In one aspect, an access assembly is disclosed including a cannula and an alignment guide. The cannula is adapted for percutaneously accessing a target tissue within a patient and includes a tubular elongate body and a handle. The tubular elongate body has a distal end, a proximal end, an outer surface and an inner luminal surface. The alignment guide includes an attachment portion and at least one radiopaque alignment indicator. The attachment portion of the alignment guide is adapted to removably attach to a portion of the outer surface of the tubular elongate body.

In another aspect, an access assembly is disclosed including a cannula and an alignment guide. The cannula is adapted for percutaneously accessing a target tissue within a patient and includes a tubular elongate body and a handle. The tubular elongate body has a distal end, a proximal end, an outer surface and an inner luminal surface. The alignment guide has an attachment portion and a radiopaque body. The attachment portion removably attaches to a portion of the outer surface of the tubular elongate body and the radiopaque body provides a visual reference of the alignment of the elongate body in relation to an axis of viewing of a radiographic device when viewed radiographically.

In another aspect a cannula for percutaneously accessing a target tissue within a patient is disclosed and includes a tubular elongate body and a handle. The tubular elongate body has a distal end, a proximal end, an outer surface and an inner luminal surface. The handle includes a first surface and a second surface separated by a thickness, where the first surface define a first plane and the second surface defines a second plane, and the first plane and second plane are substantially parallel. A first radiopaque landmark is disposed on the first surface and a second radiopaque landmark is disposed on the second surface.

In yet another aspect, a forceps assembly is adapted for grasping a tubular elongate body of a cannula. The forceps assembly includes a first member in pivotable connection with a second member, with each member having a proximal handle end and a distal grasping end. The distal grasping end of the first member and second member are pivotable between an open position and a closed position. The first member distal end includes a first grasping surface and the second member distal end having a second grasping surface, the first grasping surface and the second grasping surface are formed to grasp a portion of the tubular elongate body of the cannula in the closed position. A first radiopaque alignment indicator extends from a portion of the distal end of the first member and a second radiopaque alignment indicator extends from a portion on the distal end of the second member, such that the first radiopaque alignment indicator and the second radiopaque alignment indicator provide a visual reference of the alignment of the elongate body in relation to a target tissue when held therebetween in the closed position and viewed fluoroscopically.

In yet another aspect, an alignment guide for removably attaching to an elongate body includes an attachment portion and at least one alignment indicator. The attachment portion is adapted to removably attach to a portion of the outer surface of the elongate body and the radiopaque alignment indicator is adapted to provide a visual indication of the alignment of the cannula when viewed radiographically.

In still yet another aspect, a method of performing a medical procedure on a body is disclosed. The method includes providing a cannula assembly including a cannula adapted for percutaneously accessing a target tissue within a patient and an alignment guided. The cannula has a tubular elongate body and a handle; the tubular elongate body has a distal end, a proximal end, an outer surface and an inner luminal surface. The alignment guide has an attachment portion and at least one radiopaque alignment indicator, the attachment portion adapted to removably attach to a portion of the outer surface of the tubular elongate body. The method also includes attaching the alignment guide to a portion of the outer surface of the tubular elongate body and partially penetrating the body with the cannula. The method further includes aligning the cannula assembly with respect to a target tissue by viewing the cannula under fluoroscopy, advancing the assembled cannula system to access the target tissue, and treating the target tissue.

The present disclosure includes a number of important technical advantages. One technical advantage is that the alignment guide and alignment landmarks provide a clear indication of the relative alignment of the access assembly with respect to the viewing axis of a radiographic or similar viewing device. Another important advantage (in some embodiments) is that the alignment guide is removable and can be removed from the cannula following needle placement. This allow for the treatment area to remain uncluttered, especially when multiple needle are being placed, such as in a bipedicular procedure. Additional advantages will be apparent to those of skill in the art from the figures, description and claims provided herein.

### BRIEF DESCRIPTION OF THE DRAWINGS

The invention may best be understood by reference to the following description taken in conjunction with the accompanying drawings in which:

FIG. 1 shows a prior art cannula assembly;

FIGS. 2A and 2B show an access assembly including a detachable alignment guide according to teachings of the present disclosure;

FIGS. 3A, 3B, 3C and 3D show views of a detachable alignment guide according to teachings of the present disclosure;

FIG. 4 shows an alignment guide having a radiopaque body;

FIG. 5 shows an alignment guide having a radiopaque alignment landmark with a slide on attachment;

FIG. 6 is a representation of an access assembly in relation to a patient and a fluoroscopic viewing apparatus;

FIG. 7A shows an access assembly viewed “on-axis” by a fluoroscopic viewing apparatus;

FIG. 7B is a representation of the on-axis radiographic view of the access assembly;

FIG. 8A shows an access assembly viewed “off-axis” by a fluoroscopic viewing apparatus;

FIG. 8B is a representation of the off-axis radiographic view of the access assembly;

FIG. 9 is a representation of a procedure kit according to the present disclosure;

FIGS. 10A and 10B show a tool system accessing a vertebral body according to teachings of the present disclosure;

FIG. 11 shows a tool system for treatment of a vertebral body including a tissue treatment device according to teachings of the present disclosure;

FIGS. 12A and 12B show a tool system including an expandable device according to teachings of the present disclosure;

FIG. 13 shows a tool system for treating a vertebral body including a cement delivery system according to teachings of the present disclosure;

FIG. 14 shows forceps including an alignment guide according to teachings of the present disclosure;

FIGS. 15A-15E show an access assembly with radiographic alignment landmarks (on a handle) according to teachings of the present disclosure; and

FIG. 16 shows a method of using the present invention in a medical procedure

### DETAILED DESCRIPTION

While the present invention is described in detail, it is to be understood that this invention is not limited to particular variations set forth herein as various changes or modifications may be made to the embodiments described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The systems of the present invention may be configured for use in any suitable minimally invasive procedure that involves accessing a target site within a patient. As discussed herein, the subject systems are particularly suitable for accessing and treating vertebral bodies and long bones. In certain embodiments, the system further includes one or more treatment devices suitably configured for treating a degenerative intervertebral disc.

The treatment device of the present invention may have a variety of configurations and characteristics as described below. However, one variation of the invention employs a tissue treatment device using Coblation® technology developed by the assignee. A more detailed discussion of spinal applications and devices using Coblation® technology may be found as follows: issued U.S. Pat. Nos. 6,105,581; 6,283,961; 6,264,651; 6,277,112; 6,322,549; 6,045,532; 6,264,650; 6,464,695; 6,468,274; 6,468,270; 6,500,173; 6,602,248; 6,772,012; and 7,070,596 each of which is incorporated by reference, and pending U.S. patent applications Ser. No. 09/747,311 filed Dec. 20, 2000 and Ser. No. 10/656,597 filed Sep. 5, 2003 both of which are incorporated by reference.

The description below includes an example of the inventive method as applied to a vertebroplasty or kyphoplasty procedure. However, it is understood that the invention is not limited to vertebroplasty and kyphoplasty procedures. It is also understood that while this invention is described using fluoroscopy, other radiographic imaging systems may be used, for example CT or X-ray. The access assembly configurations disclosed herein may be used in any procedure where it is desired to establish cannular access to a bone structure or another tissue structure. Moreover, other treatment modalities (e.g., chemical, other electrosurgical devices, etc.) may be used with the inventive method either in place of Coblation® technology as discussed herein or in addition thereto.

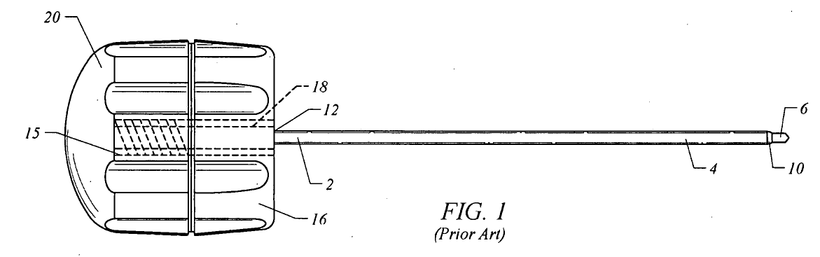

FIG. 1 shows an existing access assembly 2 that generally includes a cannula 4 and a stylet 6 operatively assembled. Stylet 6 is sized and configured for slidable insertion into and removal from cannula 4. Cannula 4 defines a hollow lumen extending from a distal end 10 to a proximal end 12. Distal end 10 may terminate in a tapered or outwardly beveled distal tip for facilitating penetration into bone or tissue of a target site. A cannula handle 16 is provided at the proximal end 12 of cannula 4 which facilitates the user's handling and manipulation of the assembly 2. Stylet handle 20 is connected with the proximal end of stylet 6. Extending proximally from handle 16 is a threaded member or connector 18 (shown in phantom in FIG. 1) for connecting with a mating connector 15 formed in stylet handle 20. Threaded member 18 may also be adapted for engagement with a system for the controlled injection of flowable material, such as polymethylmethacrylate (PMMA) based bone cement.

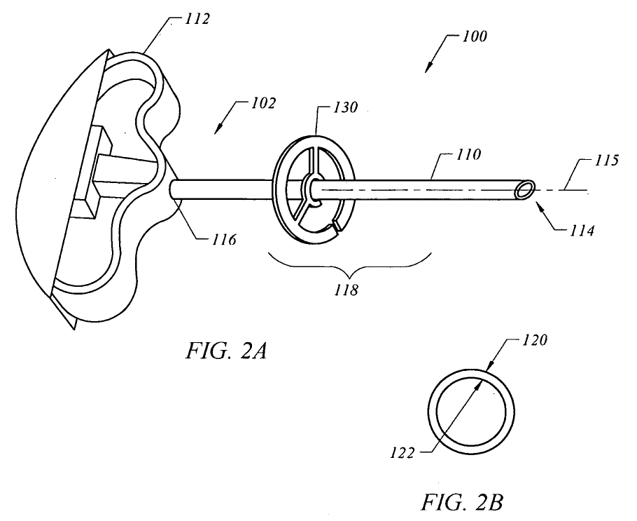

Now referring to FIG. 2A, an access assembly 100 according to the present disclosure is shown. Access assembly 100 generally includes a cannula 102 with a tubular elongate body 110 having a distal end 114, a proximal end 116 and a center section 118 therebetween. Tubular elongate body also has a longitudinal axis 115 as shown. A handle 112 is preferably connected with proximal end 116 of elongate body 110 and facilitates manipulation of cannula 102 and also allows removable entry of a stylet (not expressly shown). The present embodiment shows a representative “T-grip” type handle, however, the present invention may be employed with any suitable handle shape or configuration.

Elongate body 110 includes a generally smooth outer surface 120 and a generally smooth, substantially inviolate inner luminal surface 122 as shown in FIG. 2B. As shown in FIG. 2A, alignment guide 130 is detachably connected onto outer surface 120 of elongate body 110. As further discussed herein, when installed on elongate body 110, alignment guide 130 is generally adapted to provide a visual reference of the alignment of elongate body 110 and elongate body longitudinal axis 115 in relation to a target tissue when viewed fluoroscopically.

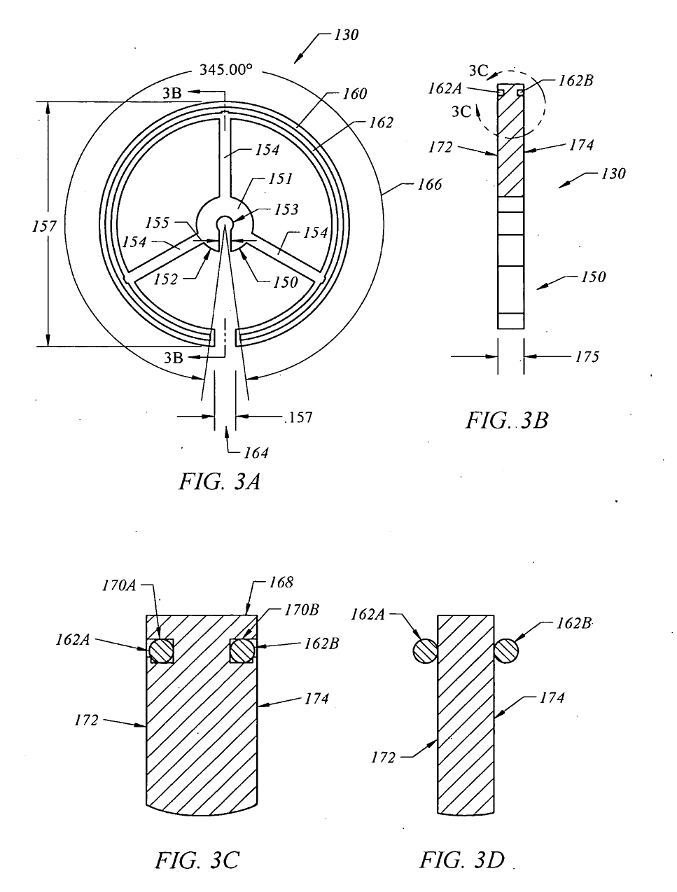

Now referring to FIG. 3A-3C, alignment guide 130 is shown. Alignment guide 130 generally includes an attachment portion 150 and an outer body 160. In the present embodiment, outer body 160 is connected with attachment portion via radial support members 154. In the present embodiment, three radial support members 154 connect attachment portion with outer body 160; in alternate embodiments, more or fewer radial support members 154 may be used. Attachment portion 150 includes hub 151 and center opening 153. Hub 151 is substantially circular and includes opening or gap 152 defined by slot 155. Attachment portion 150 and the dimensions of center opening 153 and gap 152 are preferably selected to facilitate the removable attachment of alignment guide 130 onto outer surface 120 of tubular elongate body 110. For instance, opening 152 and center opening are sized for a clip-like attachment on cannulas of a selected standard size such as, for instance a 13 gauge, 11 gauge or 8 gauge cannula or any other suitable standard cannula.

Outer member 160 is substantially circular and includes opening 164. Opening 164 is shown to be approximately 0.157 inches. In alternate embodiments opening 164 may be substantially smaller or substantially closed, provided alignment guide 130 is somewhat plastic and deformable. The user may then bend, twist or temporarily deform alignment guide 130 in order to attach it to tubular body 110. Contrarily this opening 164 may be significantly larger. In such embodiments outer member 160 may be semi circular in shape for example, or outer member arc angle 166 may be less than 270 degrees. Alignment guide 130, in this case, may be smaller in overall size and less likely to interfere during the placement of multiple access assemblies 100.

In the present embodiment, member 160, hub 151 and radial supports 154 are molded as a single component and are constructed of a relatively radiolucent and possibly biocompatible material including, but not limited to, for example, polycarbonate, various polyolefins (e.g. polyethylene, polypropylene), nylon, polystyrene, polysulfone, PEEK, ABS, are examples. Biocompatibility is only a requirement to the extent of contact with surgeon's hands—not contact with patient's internal tissues. Outer member 160 further includes at least one radiopaque alignment indicator or “landmark” 162 disposed thereon. In the embodiment of FIG. 3, radiopaque alignment landmark 162 comprises a wire or narrow strip of material disposed on the outer member 160. Alignment landmarks could be stainless steel or any other suitable radiopaque materials or compounds.

As shown in FIGS. 3B and 3C, outer member 160 includes a first surface 172 and a second surface 174 and thickness 175. First surface 172 generally defines a first plane and second surface 174 defines a second plane such that the first plane and second plane are substantially parallel with one another. A first groove 170A is formed in first surface 172 and a second groove 170B is formed in second surface 174. A first alignment landmark 162A is situated substantially in the first groove 170A and a second alignment landmark 162B is situated substantially in the second groove 170B.

FIG. 3D shows an alternative embodiment of radiopaque indicator 162 positions. Outer member 160 includes a first surface 172 and a second surface 174 similar to FIG. 3C. Alignment landmark 162A is affixed on the first surface 172 and alignment landmark 162B is affixed on second surface 174.

A further alternative embodiment, not expressly shown, includes at least one radiopaque indicator 162 inside outer member 160. Alignment guide 130 is substantially the same shape as alignment guide described in previous embodiments. In this embodiment radiopaque wires or strips of material forming a substantially circular shape may be overmolded or encapsulated within a radiolucent alignment guide 130 during the manufacturing process.

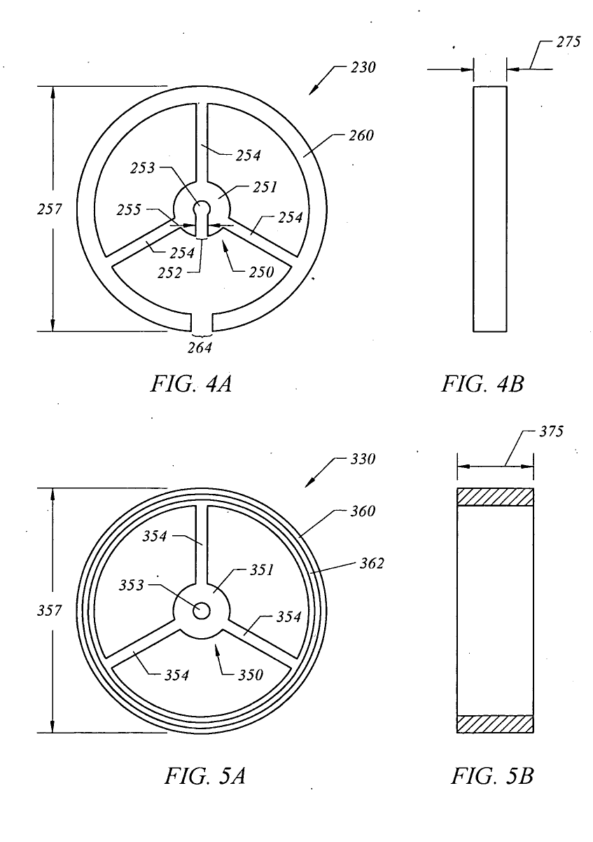

FIG. 4A shows an alignment guide 230 consisting generally of radiopaque body 260 and attachment portion 250. Radiopaque body 260 is made substantially from a radiopaque material. Only body 260 needs to be made substantially from a radiopaque material for the present invention to function as intended; the attachment portion may or may not be of substantially similar material. Material may be made radiopaque due to material properties in addition to material thickness. The thicker the material, the more visible it is fluoroscopically. Since this embodiment would provide one radiopaque landmark, the user may use two alignment guides 230 placed on the tubular body 110. Alternatively the user may use the shape of the guide 230 as a visual reference. When guide 230 is on-axis the image of the guide's shape is more circular and symmetric. Once alignment guide 230 is out of alignment the general shape is more elliptical; the image is blurred and asymmetric.

In the present embodiment, body 260 is connected with attachment portion via radial support members 254. Three radial support members 254 connect attachment portion with body 260; in alternate embodiments, more or fewer radial support members 254 may be used. Attachment portion 250 includes hub 251 and center opening 253. Hub 251 is substantially circular and includes opening or gap 252 defined by slot 255. Attachment portion 250 and the dimensions of center opening 253 and gap 252 are preferably selected to facilitate the removable attachment of alignment guide 230 onto outer surface 120 of tubular body 110. For instance, in particular embodiments opening 252 and center opening are sized for attachment on cannulas 102 of a selected standard size such as, for instance a 13 gauge, 11 gauge or 8 gauge cannula or any other suitable standard cannula.

FIG. 5 shows alignment guide 330 with an alternative attachable portion embodiment 350. This alignment guide 330 is similar to previous alignment guides 130 and 230 in spirit, and generally includes outer member 360 and radial members 354. However instead of a clip-like attachment described earlier, the present embodiment has a slide on type attachment. This attachment portion 350 consists of a substantially circular center opening 353 that slides onto elongate tubular body 110, for tubular bodies of a selected standard size such as, for instance a 13 gauge, 11 gauge or 8 gauge cannula or any other suitable standard cannula and provides sufficient friction to secure alignment guide 350 with elongate tubular body 110. This attachment portion 350 may be incorporated in any of the embodiments described herein.

In general, alignment guides 130, 230 or 330 may be any size 157, 257 or 357 and thickness 175, 275 or 375 that is practical. As guides 130, 230 or 330 have a larger diameter and thick, the more precise the use of the alignment landmarks 162, 362 may be. However to be practical, the diameter (or other longest dimension for non-circular embodiments) would not significantly exceed handle 112 dimensions so as to make it easy to use.

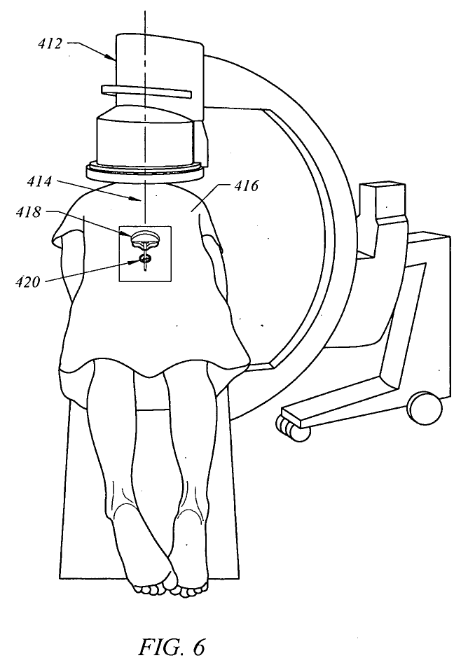

FIG. 6 represents generally how an access assembly 418 may be used in relation to a fluoroscope 412. As discussed herein, fluoroscope 412 may refer to any readiographic, fluoroscope, or other suitable medical viewing apparatus. FIG. 6 shows a patient 416 lying in a prone position with access assembly 418 inserted and alignment guide 420 attached to said access assembly 418. Fluoroscope 412 is in position above said patient 416 and above access assembly 418. Fluoroscope 412 has an axis 414 that is shown in alignment with access assembly 418.

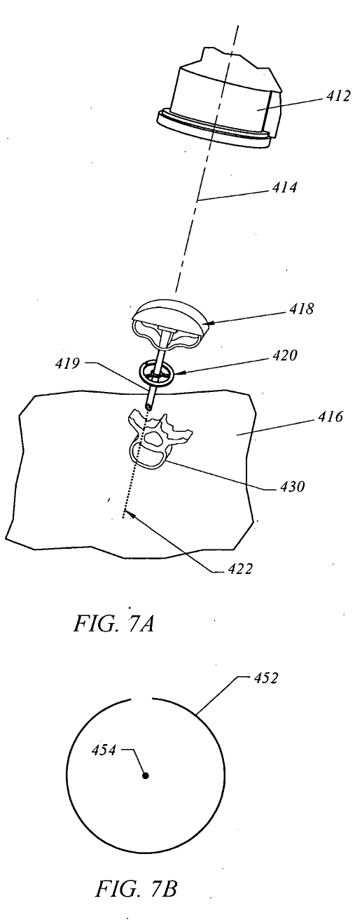

FIG. 7A shows an on-axis view of access assembly 418 with fluoroscope 412. Alignment guide 420 is shown attached to access assembly 418; in particular guide 420 is attached to tubular elongate body 419. Access assembly 418 is shown inserted into patient 416 and more specifically it is inserted into target tissue, vertebral body 430. Fluoroscope 412 is positioned above access assembly 418 and fluoroscope axis 414 is directed in-line with target tissue 430. Tubular elongate body 419 has a longitudinal axis 422 that is parallel to fluoroscope axis 414.

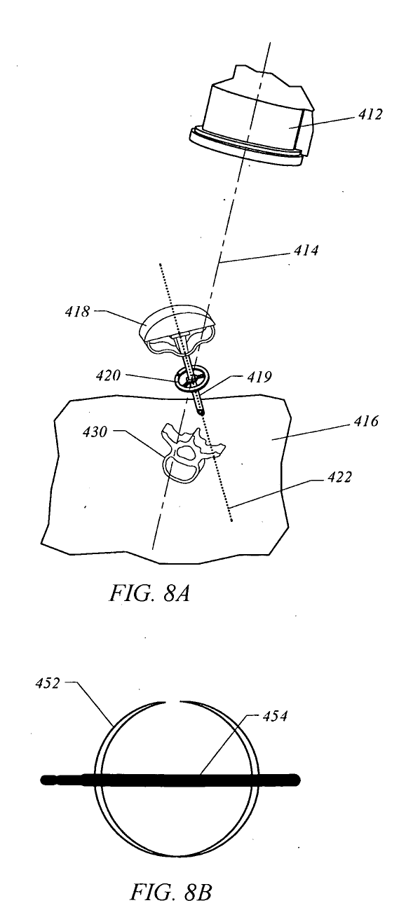

Once longitudinal axis 422 and fluoroscope axis 414 are parallel, a radiographic or fluoroscopic image similar to that shown in FIG. 7B may be viewable by the user. FIG. 7B shows a tubular elongate body portion 454 and alignment guide portion 452. Elongate body portion 454 of image will appear smallest and circular when fluoroscope axis 414 rotates to be substantially parallel to tubular body axis 422. Once in alignment, alignment landmark portion of image 452 appears substantially circular. In embodiments using more than one alignment landmarks such as shown in FIGS. 3A-3D, the user may preferably see one coincidental substantially circular shape. FIG. 8B described below will distinguish this FIG. 7B, from an image where fluoroscope axis 412 is not substantially parallel to tubular longitudinal axis 422.

FIG. 8A shows access assembly 418 which is not in axial alignment with respect to fluoroscope 412. Alignment guide 420 is shown attached to access assembly 418; in particular guide 420 is attached to tubular elongate body 419. Access assembly 418 is positioned for insertion into patient 416. Fluoroscope 412 is positioned above and fluoroscope axis 414 is directed at target tissue, vertebral body 430. Tubular elongate body 419 has a longitudinal axis 422 that is unparallel to fluoroscope's axis 414 and, in the present depiction, is not properly aligned a pedicular insertion in target tissue, vertebral body 430.

When access assembly axis 422 and fluoroscope axis 414 are not substantially parallel, fluoroscope 412 may show an image similar to that shown in FIG. 8B. This image consists of tubular elongate body portion 454 and alignment guide portion 452. Elongate body portion 454 of the image will appear elongated or oblong when fluoroscope axis 414 is not substantially parallel with tubular body axis 422. This body portion 454 image may be part of the length of tubular body 419. The alignment landmark portion of image 452 appears predominantly elliptical and asymmetric and two distinct shapes or rings may preferably exhibit a parallax effect when fluoroscope axis 414 is substantially not parallel to tubular body axis 422. As access assembly axis 422 and fluoroscope axis 414 are moved closer to a parallel position, the image of body portion 454 become correspondingly shorter and more circular and the parallax image of alignment guide portion is narrowed and also becomes more circular (e.g. the gap between the two rings closes until only a single circular element is perceived). In this manner, the user can ad just the position of the access assembly and/or the imaging device until a desired alignment is achieved.

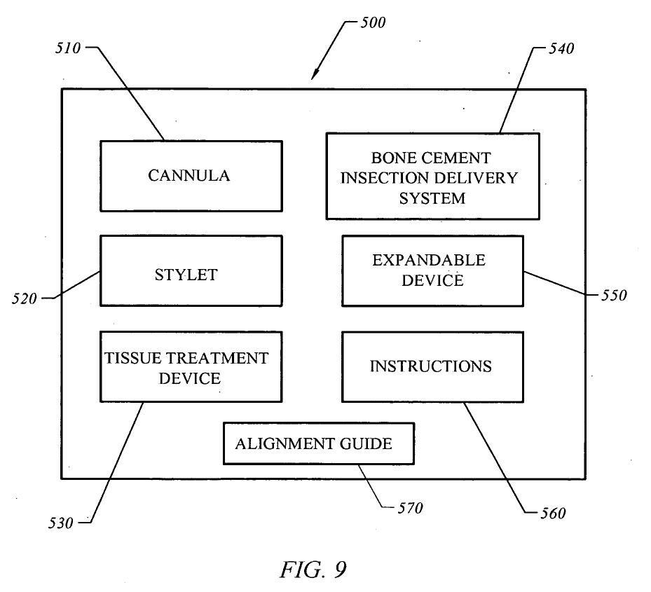

Now referring to FIG. 9, a kit, depicted generally at 500, may include component devices to perform the medical procedures as described above. FIG. 9 is a block diagram representation of a kit 500 in accordance with an embodiment of the present invention. A kit 500 may include, but is not limited to including, at least one cannula 510, at least one stylet 520, a tissue treatment device 530, a bone cement injection delivery system 540, an expandable device 550 and at least one alignment guide 570. It should be appreciated that multiple cannulas 510 and stylets 520 may be provided for use in a variety of applications. Also, in alternate kit embodiments 500 may be provided without, for example, a tissue treatment device 530 and/or an expandable device 550.

A subject kit such as kit 500 typically may preferably include instructions 560 and other pertinent documentation for using the subject systems, e.g., cannula 510 and stylet 520, in methods according to the subject invention. Instructions 560 for practicing the subject methods are generally recorded on a suitable recording medium. For example, the instructions may be printed on a substrate, such as paper or plastic, etc. As such, instructions 560 may be present in the kits as a package insert or in the labeling of the container of the kit or components thereof, i.e., associated with the packaging or subpackaging. In other embodiments, instructions 560 may include electronic data stored on a suitable computer readable storage medium, e.g., CD-ROM, DVD, diskette, etc. In yet other embodiments, the actual instructions are not present in the kit, but means for obtaining the instructions from a remote source, e.g., via the Internet, are provided in lieu of instructions 560. An example of this embodiment is a kit that includes a web address where the instructions may be viewed and/or from which the instructions may be downloaded. As with the instructions, this means for obtaining the instructions is recorded on a suitable substrate.

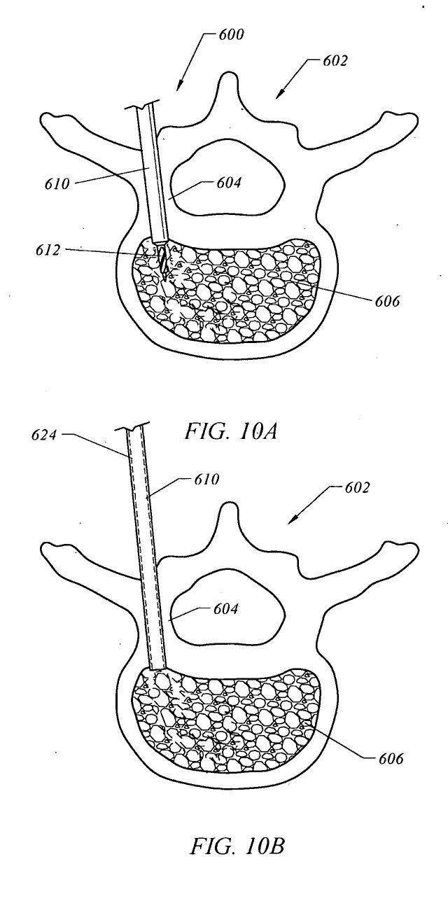

Now referring to FIG. 10A, a depiction of a tool system 600 (with portions removed) is shown entering a bone structure 602 to perform a medical procedure in accordance with teachings of the present disclosure. In the present embodiment bone structure 602 is a vertebral body, however, in alternate embodiments tool system 600 may be used to access other body structures such as the epiphysis, metaphysis, or diaphysis, of long bones, the subchondral bone of the tibia, femur, or humerus, and the pelvis, calcaneus, sacrum, and cranium. Vertebral body 602 includes cancellous tissue 606 and cortical bone, including pedicle 604. As depicted in the present embodiment, tool system 600 includes cannula body 610 and stylet 612.

In the present embodiment tool system 600 is advanced until the distal end of cannula 610 accesses cancellous bone 606. Following the desired placement of tool system 600 within bone structure 602, stylet 612 is preferably removed, opening lumen 624 within cannula 610 and thereby providing access to cancellous tissue 606, as shown in FIG. 10B.

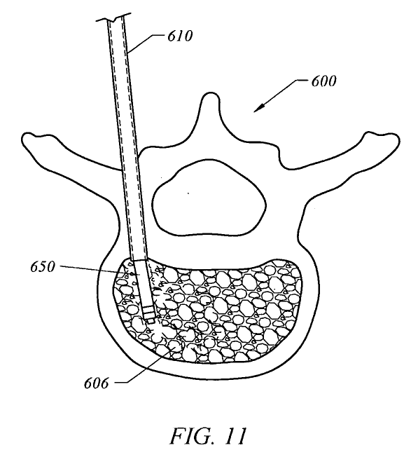

Now referring to FIG. 11, a depiction of a tool system 600 being used in a medical procedure according to teachings of the present disclosure is shown. In the present embodiment, initial cannula placement has been achieved as described above. In the present depiction, a treatment device 650 is introduced through cannula 610 to access cancellous tissue 606. In one embodiment, treatment device 650 may be a plasma-based treatment device incorporating Coblation technology as discussed above. In a particular embodiment, treatment device 650 may be a treatment device as described in U.S. patent application Ser. No. 10/970,796 which is hereby incorporated by reference. Treatment device 650 may also include a lumen for providing fluid, such as a conductive fluid as well as a suction lumen for removing said fluid and treated tissue (not expressly shown). In some embodiments, the target tissue and/or bone structure may include tumor tissue and cannula 610 may be placed to access and treat portions of said tumor tissue.

In alternate embodiments treatment device 650 may comprise any mechanical or electrosurgical device suitable for treating target tissue such as cancellous tissue 606 or tumor tissue existing within a bone structure. Treatment of target tissue may include, but is not limited to, heating, cutting, ablating and removing the target tissue.

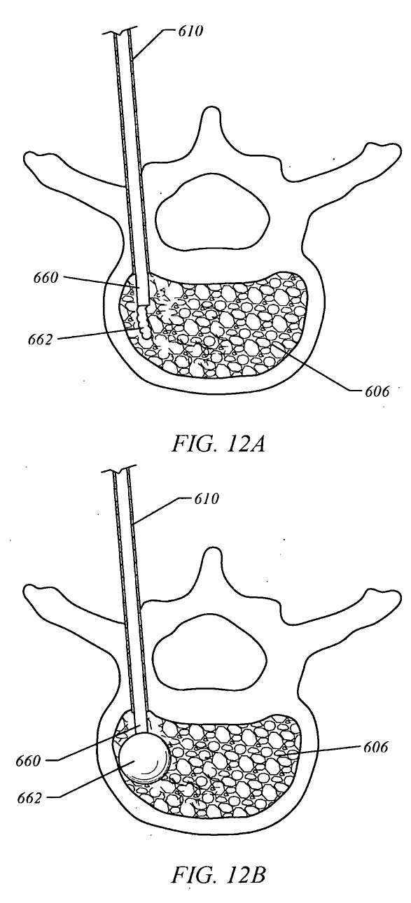

Now referring to FIG. 12A, the introduction of an expandable device 662 during a medical procedure according to teachings of the present disclosure is shown. Following placement of cannula 610 and removal of the stylet or obturator as described above, a delivery tool 660 may be inserted into cannula 610. Delivery tool 660 may be used to place expandable structure 662, within cancellous bone 606. In some embodiments, delivery tool 660 or a separate tool may be adapted to create a void in cancellous bone 606 to facilitate initial placement of the expandable structure 662 in a collapsed configuration.

Following initial placement of expandable structure 662, expandable structure is expanded, as shown in FIG. 12B. The expansion of structure 662 may be accomplished by introducing fluid into expandable structure 662 up to a selected pressure. As structure 662 expands, portions of cancellous bone 606 adjacent to the expandable structure are pushed away from expanding structure 662. Expandable structure 662 may be collapsed and removed via cannula 610, leaving a void within cancellous bone 606.

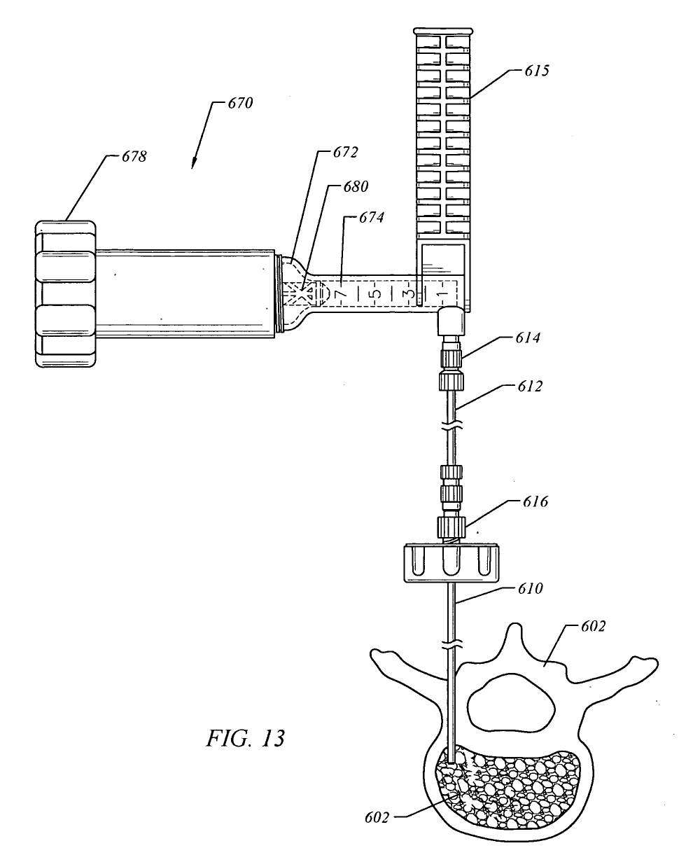

Following initial placement of cannula 610, the treatment of cancellous tissue 606 and/or the use of an expandable device 662, cement or filler material may be introduced into the target site of bone structure 602, as shown in FIG. 13. In such procedures, stylet 612 may preferably be removed from cannula 610 which is left in place at the target site. A system 670 for the controlled injection of filler material is operatively coupled to cannula 610, as shown in FIG. 13, so as to be in fluid communication with the cannula's lumen. System 670 generally includes a first column 672 and a second column 674 which holds the filler material. A handle 678 at the proximal end of first column 672 is used turn first column 672 to drive the filler material into the second column 674 where the plunger head can pressurize the filler material. Extending distally from handle 678 is a plunger head 680 for forcing the filler material through the second column 674. System 670 is in fluid communication with cannula 610 by means of a tubing 612 which is interconnected to system 670 and cannula 610 by luer locks 614 and 616, respectively. In some embodiments, tubing 612 may be a flexible conduit having sufficient length to remove users hands from a radiographic field centered at cannula 610. A handle 615 is provided for manually handling system 670. Once system 670 is properly connected to cannula 610, the filler material is delivered to within the space created by treatment device 650 or expandable device 662 described above until a selected amount of such filler material has been injected into the space. Upon completion of the filling process, the system 670 is disconnected from cannula 610 which may then be removed from the access site, and the wound site is treated with typical care.

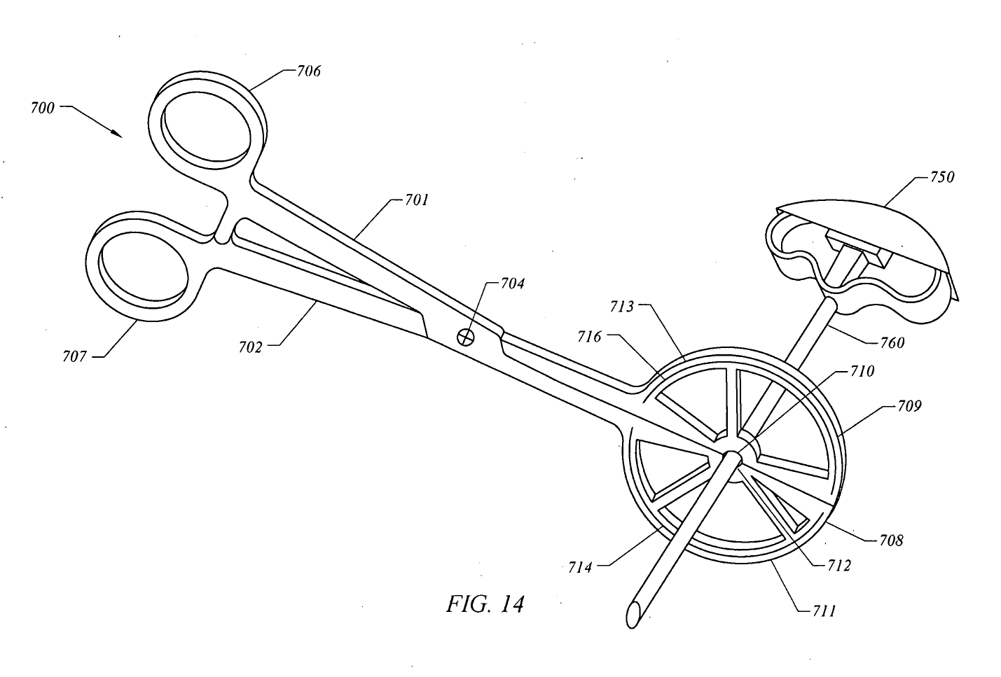

FIG. 14 shows cannula 750 grasped by forceps assembly 700. Forceps assembly includes first member 701 and second member 702 that are pivotally attached at pivotable connection 704. First member 701 includes a proximal handle end 706 and a distal grasping end 708. Second member 702 includes a proximal handle end 707 and distal grasping end 709. By rotating proximal handle ends 706 and 707 relative to each other, forceps assembly 700 may be moved from an open to a closed position. At distal grasping ends 708 and 709, there are corresponding first and second distal grasping surfaces 710 and 712. When forceps assembly 700 is in a closed position, first and second grasping surfaces 710 and 712 grasp a portion of an elongate body 760 of cannula 750 such that the distal end of forceps assembly is held substantially perpendicular to elongate body 760. Once grasped, cannula 750 may be rotated and oriented to be on axis with a fluoroscope axis using forceps assembly 700.

As shown, a first alignment guide portion 711 extends from first distal end 708 and a second alignment guide portion 713 extends from second distal 709. Each alignment guide portion 711 and 713 is substantially semicircular in shape and includes a radiopaque alignment indicator portion 714 and 716 towards the outer circumference of each guide 711 and 713.

Once the forceps assembly 700 is in a closed position, these indicator portions 714 and 716 form an approximate circular shape. In alternate embodiments, the distal end of forceps assembly, when in a closed position may generally approximate the features of any of the alignment guide embodiments described in FIG. 3, 4 or 5.

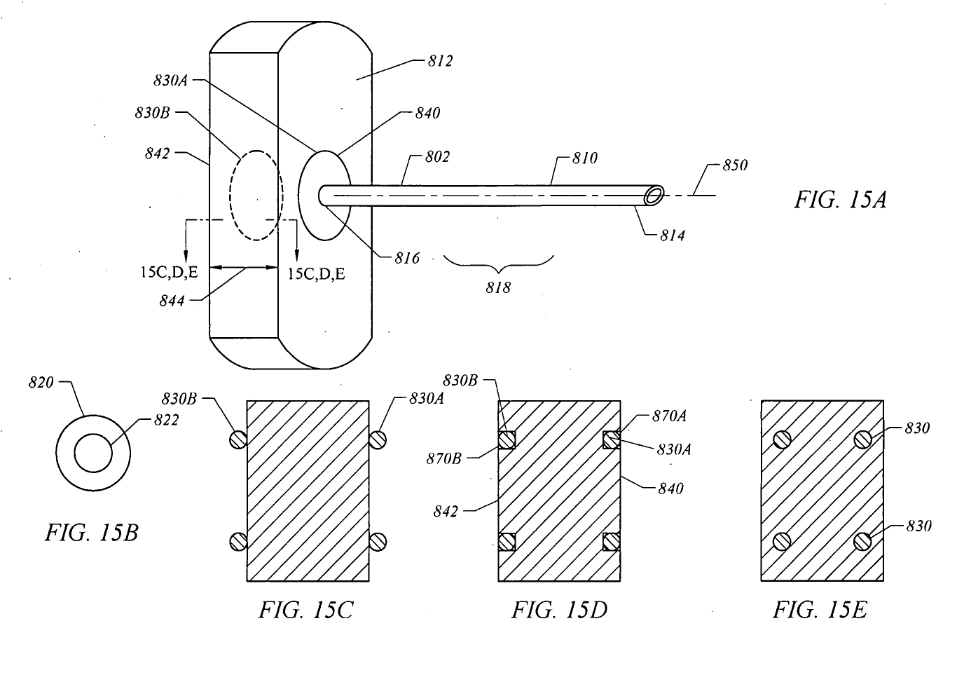

Now referring to FIG. 15, a cannula 802 according to the present disclosure is shown. Cannula 802 generally includes a tubular elongate body 810 having a distal end 814, a proximal end 816 and a center section 818 therebetween and a handle 812, which is preferably connected with proximal end 816 and facilitates manipulation of cannula 802. As shown in FIG. 15B, elongate body 810 generally has an outer surface 820 and an inner luminal surface 822.

Handle 812 includes a first surface 840 and second surface 842, separated by thickness 844. First surface 840 and second surface 842 are substantially parallel to each other and substantially perpendicular to elongate tubular body longitudinal axis 850. A first radiopaque landmark 830A is disposed on said first surface 840 and a second radiopaque landmark 830B is disposed on said second surface 842. Both landmarks 830 are substantially circular in shape, and first landmark 830A is concentric with 830B. Landmarks 830 do not need to be a complete circle, but enough of a circle to make landmarks 830 easy to visualize on an imaging system. Landmark 830A is shown on FIG. 15A to have substantially the same diameter as landmark 830B. Landmarks 830 may be concentric and different diameters. In alternate embodiments, landmarks 830A and 830B may be in any shape or combination of shapes suitable for showing the relative alignment of the landmarks with respect to a viewing axis of a radiographic or similar viewing device. For example, landmarks may be of different sizes and shapes such that one landmark may “nest” within the second landmark when properly aligned or one landmark may display crosshairs which may be aligned with a second landmark.

Handle 812 material is constructed of a relatively radiolucent material. Landmarks 830 may be constructed from stainless steel of any other suitable radiopaque material or compound and comprised of a wire or narrow strip of material disposed on or in said handle 812. FIG. 15C shows a cross section of handle 812 with landmarks 830 disposed on said surfaces.

As shown in FIG. 15D, first groove 870A is formed in first surface 840 and a second groove 870B is formed in second surface 842. A first alignment landmark 830A is situated substantially in first groove 870A and a second alignment landmark 830B is situated substantially in second groove 870B.

Another alternative embodiment, shown in FIG. 15E, includes at least one radiopaque indicator 830 inside handle 812. In this embodiment radiopaque wires or strips of material forming a substantially circular shape are overmolded by radiolucent materials during the manufacturing process. In other alternate embodiments using overmolding as described, handle 812 may take on a shape in which the opposing surfaces of handle 812 may have contoured or ergonomically designed surfaces that are not parallel (not expressly shown).

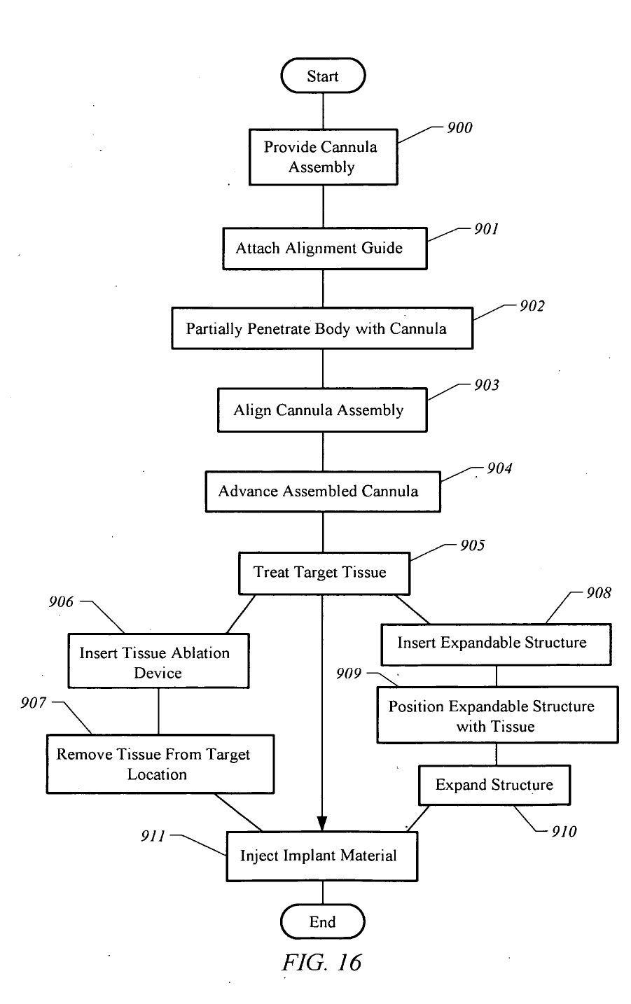

A method of performing a medical procedure using alignment guide is shown in FIG. 16. A cannula assembly is first provided 900, for accessing target tissue and an alignment guide is attached to the cannula assembly 901, on a tubular elongate portion of said cannula. The cannula is then inserted partially into the patient body 902 towards the target tissue. Using imaging means and the alignment guide, the cannula assembly is aligned with respect to the target tissue 903. The cannula assembly is then advanced more towards the target tissue 904. Once the cannula is inside the target tissue, the tissue is treated 905.

Treating the tissue may include inserting a distal end of a tissue ablation device 906 in the cannula and removing tissue from the target location 907.

Treating the target tissue may include inserting an expandable structure in the cannula 908, positioning the expandable structure within the target tissue 909 and causing the expandable structure to assume an expanded geometry 910.

The target tissue may be a portion of cancellous bone within a vertebral body and treating this tissue may include injecting a settable implant material 911 into the cancellous bone.

Although only a few embodiments of the present invention have been described, it should be understood that the present invention may be embodied in many other specific forms without departing from the spirit or the scope of the present invention. Therefore, the present examples are to be considered as illustrative and not restrictive, and the invention is not to be limited to the details given herein, but may be modified within the scope of the appended claims.

What is claimed is:

1. An access assembly comprising:
a cannula adapted for percutaneously accessing a target tissue within a patient, the cannula having a tubular elongate body and a handle;
the tubular elongate body having a distal end, a proximal end, an outer surface and an inner luminal surface; and
an alignment guide having an attachment portion and at least one radiopaque alignment indicator, the attachment portion adapted to removably attach to a portion of the outer surface of the tubular elongate body.

2. The assembly of claim 1 wherein the radiopaque alignment indicator is adapted to provide a visual reference of the alignment of the elongate body in relation to the target tissue when viewed radiographically.

3. The assembly of claim 1 wherein the alignment guide comprises a radiopaque substantially circular outer member connected with the attachment portion.

4. The assembly of claim 3 further comprising at least one radial support member connecting the attachment portion and the outer member.

5. The assembly of claim 1 wherein:
the alignment guide comprises a body having a first surface and a second surface separated by a thickness, the first surface defining a first plane and the second surface defining a second plane, the first plane substantially parallel to the second plane; and
the radiopaque alignment indicator comprises a first radiopaque alignment landmark disposed on the first surface and a second radiopaque alignment landmark disposed on the second surface.

6. The assembly of claim 5 wherein the first radiopaque alignment landmark is substantially concentric and similar in diameter with the second radiopaque alignment landmark.

7. The assembly of claim 1 wherein
the alignment guide comprises a first surface and a second surface separated by a thickness, the first surface defining a first plane and the second surface defining a second plane, the first plane substantially parallel to the second plane, a first groove formed in the first surface and a second groove formed in the second surface; and
the radiopaque alignment indicator comprises a first radiopaque alignment landmark disposed in the first groove and a second radiopaque alignment landmark disposed in the second groove.

8. The assembly of claim 7 wherein the radiopaque alignment landmark comprises a wire.

9. The assembly of claim 7 wherein the second groove is formed substantially concentric and similar in diameter to the first groove.

10. The assembly of claim 5 wherein the first radiopaque landmark and the second radiopaque landmark are adapted to provide a visual reference of the alignment of the elongate body in relation to the target tissue when viewed fluoroscopically.

11. The assembly of claim 5 wherein the first radiopaque landmark and the second radiopaque landmark are adapted to provide a visual reference of the alignment of the elongate body in relation to an axis of viewing of a radiographic device when viewed radiographically.

12. The assembly of claim 1 wherein the attachment portion is adapted to attach the alignment guide substantially perpendicular to a longitudinal axis of the tubular elongate body.

13. The assembly of claim 1 wherein the attachment portion is adapted to clip to the outer surface of the tubular attachment body.

14. The assembly of claim 1 wherein the attachment portion is adapted to slide on to the outer surface of the tubular attachment body.

15. The assembly of claim 1 further comprising a stylet adapted to fit within and be removed from the tubular elongate member.

16. The assembly of claim 1 wherein the target tissue comprises cancellous bone.

17. The assembly of claim 1 wherein the target tissue comprises cancellous bone within a vertebral body.

18. An access assembly comprising:
a cannula adapted for percutaneously accessing a target tissue within a patient, the cannula having a tubular elongate body and a handle;
the tubular elongate body having a distal end, a proximal end, an outer surface and an inner luminal surface; and
an alignment guide having an attachment portion and a radiopaque body, the attachment portion adapted to removably attach to a portion of the outer surface of the tubular elongate body, the radiopaque body adapted to provide a visual reference of the alignment of the elongate body in relation to an axis of viewing of a radiographic device when viewed radiographically.

19. A cannula for percutaneously accessing a target tissue within a patient comprising:
a tubular elongate body having a distal end, a proximal end, an outer surface and an inner luminal surface; and
the handle comprising a first surface and a second surface separated by a thickness, the first surface defining a first plane and the second surface defining a second plane, the first plane substantially parallel to the second plane, a first radiopaque landmark disposed on the first surface and a second radiopaque landmark disposed on the second surface.

20. The cannula of claim 19 where the second radiopaque landmark is substantially concentric and of similar diameter to the first radiopaque landmark.

21. The cannula of claim 19 further comprising a first groove formed in the first surface and a second groove formed in the second surface, the first radiopaque landmark disposed in the first groove and the second radiopaque landmark disposed in the second groove.

22. The cannula of claim 19 wherein the first radiopaque landmark and the second radiopaque landmark are positioned to provide a visual reference of the alignment of a longitudinal axis of the elongate body with the viewing axis of a fluoroscopic device when viewed fluoroscopically.

23. A forceps assembly adapted for grasping a tubular elongate body of a cannula comprising:
a first member in pivotable connection with a second member, each member having a proximal handle end and a distal grasping end;
the distal grasping end of the first member and second member pivotable between an open position and a closed position;
the first member distal end having a first grasping surface and the second member distal end having a second grasping surface, the first grasping surface and the second grasping surface adapted to grasp a portion of the tubular elongate body of the cannula in the closed position;
a first radiopaque alignment indicator extending from a portion of the distal end of the first member and a second radiopaque alignment indicator extending from a portion on the distal end of the second member, the first radiopaque alignment indicator and the second radiopaque alignment indicator adapted to provide a visual reference of the alignment of the elongate body in relation to a target tissue when held therebetween in the closed position and viewed fluoroscopically.

24. An alignment guide for removably attaching to an elongate body comprising:
an attachment portion adapted to removably attach to a portion of the outer surface of the elongate body; and
at least one radiopaque alignment indicator adapted to provide a visual indication of the alignment of the cannula when viewed radiographically.

25. The alignment guide of claim 24 wherein the alignment guide comprises a substantially circular outer member connected with the attachment portion.

26. The alignment guide of claim 24 further comprising:
a body having a first surface and a second surface separated by a thickness, the first surface defining a first plane and the second surface defining a second plane, the first plane substantially parallel to the second plane; and
wherein the radiopaque alignment indicator comprises a first radiopaque alignment landmark disposed on the first surface and a second radiopaque alignment landmark disposed on the second surface.

27. The alignment guide of claim 26 wherein the first radiopaque alignment landmark is concentric and substantially the same diameter as the second radiopaque alignment landmark.

28. The alignment guide of claim 27 wherein the first radiopaque alignment landmark comprises a wire.

29. The alignment guide of claim 24 wherein the radiopaque alignment indicator is adapted to provide a visual reference of the alignment of the cannula with an axis of viewing of a fluoroscopic device when viewed fluoroscopically.

30. The alignment guide of claim 24 wherein the attachment portion is adapted to attach the alignment guide substantially perpendicular to a longitudinal axis of the cannula.

31. A medical procedure to be performed on a body comprising:
providing a cannula assembly comprising:
a cannula adapted for percutaneously accessing a target tissue within a patient, the cannula having a tubular elongate body and a handle, the tubular elongate body having a distal end, a proximal end, an outer surface and an inner luminal surface; and
an alignment guide having an attachment portion and at least one radiopaque alignment indicator, the attachment portion adapted to removably attach to a portion of the outer surface of the tubular elongate body;
attaching the alignment guide to a portion of the outer surface of the tubular elongate body;
partially penetrating the body with the cannula;
aligning the cannula assembly with respect to a target tissue by viewing the cannula under fluoroscopy;
advancing the assembled cannula system to access the target tissue; and
treating the target tissue.

32. The medical procedure of claim 31 wherein treating the target tissue comprises:
inserting a distal end of a tissue ablation device in the cannula; and
removing tissue from the target location.

33. The medical procedure of claim 31 wherein treating the target tissue comprises:
inserting an expandable structure in the cannula;
positioning the expandable structure within the target tissue; and
causing the expandable structure to assume an expanded geometry.

34. The medical procedure of claim 31 wherein the target tissue comprises a portion of cancellous bone within a vertebral body and the step of treating the target tissue comprises injecting a settable implant material into the cancellous bone.

35. A kit for treating a vertebral body comprising:
a cannula;
a stylet adapted to fit within and be removed from said cannula;
an alignment guide having an attachment portion and at least one radiopaque alignment indicator, the attachment portion adapted to removably attach to a portion of the outer surface of the cannula, the radiopaque alignment indicator adapted to provide a visual indication of the alignment of the cannula when viewed flouroscopically; and
a settable implant injection delivery system having a connector adapted to fluidly connect said delivery system to said cannula to deliver a settable implant material to a target site

36. The kit of claim 35 further comprising an expandable device adapted to be delivered to the target site through the cannula and expanded within the target site.

37. The kit of claim 35 further comprising a tissue removal device having a distal end adapted to fit within the cannula and remove tissue at the target site.

38. An access assembly comprising:
a cannula adapted for percutaneously accessing a target tissue within a patient, the cannula having a tubular elongate body and a handle;
the tubular elongate body having a distal end, a proximal end, an outer surface and an inner luminal surface; and
an alignment means having an attachment means for removably attaching to a portion of the outer surface of the tubular elongate body and at least one radiopaque alignment means for indicating the alignment of the cannula with an axis of viewing of a fluoroscopic viewing device when viewed fluoroscopically.
